(19) United States
(12) Patent Application Publication
(10) Pub. No.: US 2004/0111788 A1
(43) Pub. Date: Jun. 17, 2004

(54) Composite len for swimming or diving goggles

(76) Inventor: Terry Chou, Tainan City (TW)

Correspondence Address: Charles E. Baxley, Esquire, 5th Floor, 59 John Street, New York, NY, 10038, US

(21) Appl. No.: 10/316,530

(22) Filed: Dec. 12, 2002

(51) Int. Cl. A61F 9/02

(52) U.S. Cl. 2/426

(57) ABSTRACT

A pair of goggles includes a flexible frame having an annular groove and a composite lens. The composite lens includes a plurality of lenses that are overlapped one on another. An annular bottom wall defining the annular inner groove of the flexible frame is bonded with a periphery of each lens as an integral unit. In another embodiment, the pair of goggles includes a frame having an annular groove, a composite lens, and a soft padding member. The composite lens includes a plurality of lenses that are overlapped one on another. The composite lens has a periphery received in the frame. The soft padding member has a portion mounted between a periphery of the composite lens and the frame. The soft padding member is bonded with the frame as an integral unit. The soft padding member is bonded with a periphery of each lens as an integral unit

### BACKGROUND OF THE INVENTION

1. Field of the Invention

The present invention relates to a composite lens for swimming or diving goggles with improved convenience to assembling procedure, improved water-resistance, and improved stability.

2. Description of the Related Art

A pair of conventional goggles for swimming or diving includes at least one frame and a single lens held in each frame. The single lens provides only one optical function. To provide additional functions, a composite lens is provided in conventional swimming or diving goggles. FIG. 10 of the drawings illustrates a conventional composite lens comprised of a first lens 1′ and a second lens 2′ adhered to a side of the first lens 1′ by adhesive 3′. The first lens 1′ and the second lens 2′ are of different colors or have different functions such as filtration and polarization. However, the adhesive 3′ between the first lens 1′ and the second lens 2′ adversely affect the vision. Further, the adhesive 3′ deteriorates and thus adversely affects the transparency of the adhesive 3′. In some cases, the adhesive 3′ losses its adhesion and thus causes separation of the first lens 1′ and the second lens 2′. Further, as illustrated in FIGS. 11 and 12, since the first lens 1′ and the second lens 2′ are made from different materials, the amount of deformation of the first lens 1′ differs from that of the second lens 2′ when subject to external force or the ambient temperature changes, causing separation of the first lens 1′ and the second lens 2′. As a result, water is apt to enter the gap resulting from the separation and thus causes vague vision.

FIGS. 13 and 14 illustrate another conventional composite lens comprised of a first lens 4′ and a second lens 5′ that are made from different materials. The first lens 4′ includes a recessed portion 41′. The second lens 5′ is attached to a side of the first lens 4′. An ultrasonic welding procedure is carried out along a peripheral edge portion between the first lens 4′ and the second lens 5′ to thereby bond the first lens 4′ and the second lens 5′ together. The first lens 4′ further includes an outer flange 42′ to be inserted into a groove 61′ of a frame 6′. However, an ultrasonic welding procedure is required. Further, as shown in FIG. 15, the first lens 4′ and the second lens 5′ made from different materials tend to separate from each other at the joining area “a”. As a result, the vision is adversely affected, as water could enter the space between the first lens 4′ and the second lens 5′. Further, water is still apt to enter an interior of the frame 6′ via a jointing gap existed between the composite lens and the frame 6′, as the composite lens is only engaged with the groove 61′ of the frame 6′ by the outer flange 42′.

### SUMMARY OF THE INVENTION

An object of the present invention is to provide a composite lens for swimming or diving goggles with improved convenience to assembling procedure, improved water-resistance, and improved stability.

In an embodiment of the invention, a pair of goggles in accordance with the present invention includes a flexible frame having an annular groove and a composite lens. The annular groove is defined by an annular bottom wall, an annular inner wall, and an annular outer wall. The composite lens includes a plurality of lenses that are overlapped one on another. The annular bottom wall defining the annular inner groove of the flexible frame is bonded with a periphery of each of the plurality of lenses as an integral unit.

In another embodiment of the invention, a pair of goggles includes a frame having an annular groove, a composite lens, and a soft padding member. The composite lens includes a plurality of lenses that are overlapped one on another. The composite lens has a periphery received in the frame. The soft padding member has a portion mounted between a periphery of the composite lens and the frame. The soft padding member is bonded with the frame as an integral unit. The soft padding member is bonded with a periphery of each of the plurality of lenses as an integral unit.

Other objects, advantages, and novel features of the invention will become more apparent from the following detailed description when taken in conjunction with the accompanying drawings.

### BRIEF DESCRIPTION OF THE DRAWINGS

FIG. 1 is an exploded perspective view of a first embodiment of a composite lens in accordance with the present invention.

FIG. 2 is a sectional view of a portion of a pair of goggles with the composite lens in FIG. 1

FIG. 3 is a sectional view similar to FIG. 2, illustrating deformation of the composite lens.

FIG. 4 is a sectional view similar to FIG. 2, illustrating a second embodiment of the composite lens in accordance with the present invention.

FIG. 5 is an exploded perspective view of a third embodiment of the composite lens in accordance with the present invention.

FIG. 6 is a sectional view of a frame and the composite lens in FIG. 5.

FIG. 7 is a sectional view similar to FIG. 7, illustrating formation of a padding member between the frame and the composite lens in FIG. 6.

FIG. 8 is a sectional view similar to FIG. 7, illustrating a fourth embodiment of the composite lens in accordance with the present invention.

FIG. 9 is a sectional view similar to FIG. 8, illustrating a fifth embodiment of the composite lens in accordance with the present invention.

FIG. 10 is a sectional view of a conventional composite lens.

FIG. 11 is a sectional view similar to FIG. 10, illustrating separation of the composite lens.

FIG. 12 is a conventional view illustrating separation of the composite lens in FIG. 11.

FIG. 13 is a sectional view of another conventional composite lens.

FIG. 14 is a sectional view illustrating use of the composite lens in FIG. 13 with a frame of a pair of swimming goggles.

FIG. 15 is a sectional view similar to FIG. 14, illustrating leakage of the composite lens in FIG. 14.

### DETAILED DESCRIPTION OF THE PREFERRED EMBODIMENTS

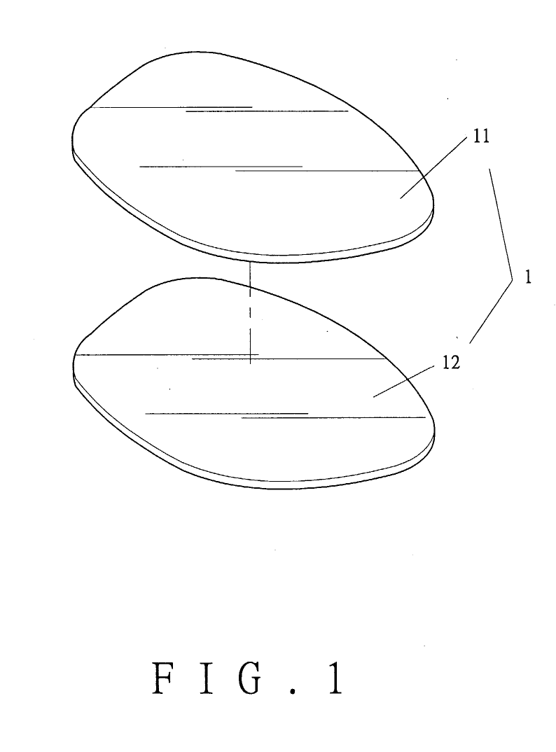

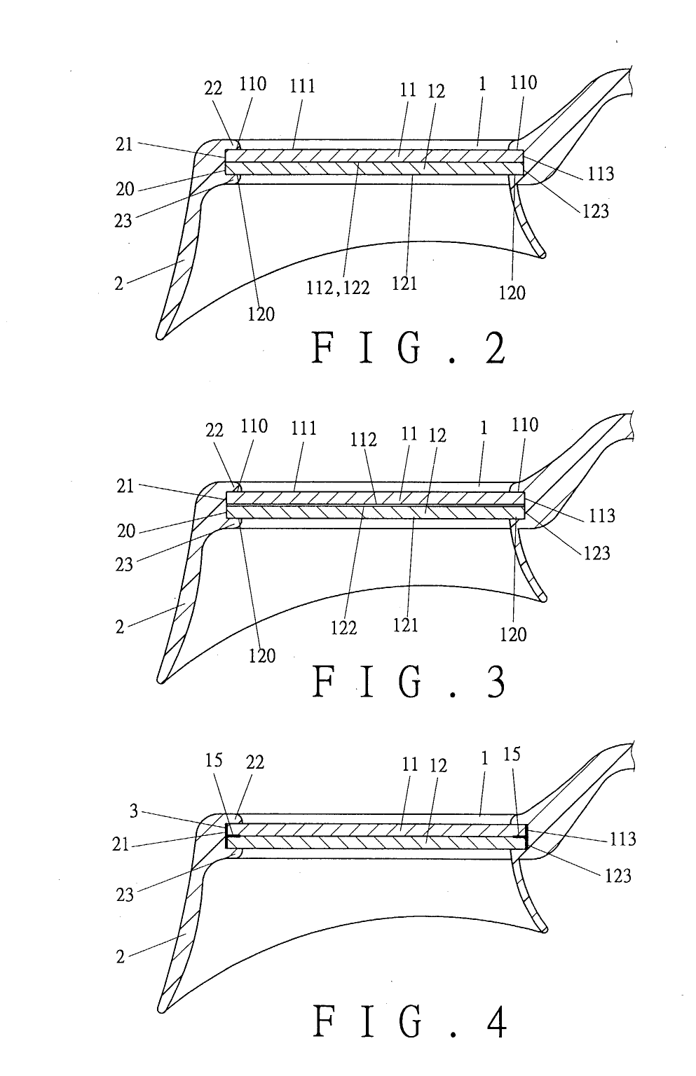

Referring to FIGS. 1 and 2, a composite lens 1 in accordance with the present invention generally comprises at least two lenses made from different materials. In a first embodiment shown in FIGS. 1 and 2, the composite lens 1 includes a first, outer lens 1 and a second, inner lens 12 that are overlapped one on the other. In formation, the first lens 11 and the second lens 12 are placed with an inner side 112 of the outer lens 11 being in face-to-face contact with an outer face 122 of the inner lens 12. The composite lens 1 is then placed into a mold (not shown). Plastic material is poured into the mold for forming a flexible frame 2 that includes an annular inner groove 20. An annular bottom wall 21 defining the annular inner groove 20 of the flexible frame 2 is bonded with a periphery 113 of the outer lens 11 and a periphery 123 of the inner lens 12. Further, an annular outer wall 22 defining the annular inner groove 20 of the flexible frame 2 is bonded with an outer annular edge portion 110 on an outer side 111 of the outer lens 11. Further, an annular inner wall 23 defining the annular inner groove 20 of the flexible frame 2 is bonded with an inner annular edge portion 120 on an inner side 121 of the inner lens 12. Thus, the composite lens 1 is bonded with the walls 21, 22 and 23 defining the inner groove 20 of the flexible frame 2 as an integral unit. The pair of goggles for swimming or diving thus formed provides good vision, as there is no adhesive between the outer lens 11 and the inner lens 12.

Referring to FIG. 3, since the outer lens 11 and the inner lens 12 constituting the composite lens 1 are made from different materials, the amount of deformation of the outer lens 11 differs from that of the inner lens 12. As a result, a gap is created between the outer lens 11 and the inner lens 12 when ambient temperature changes. Nevertheless, the composite lens 1 still provides a good vision, as there is no adhesive between the outer lens 11 and the inner lens 12. Further, the annular outer wall 22 defining the annular inner groove 20 of the flexible frame 2 is still bonded to the outer annular edge portion 110 on the outer side 111 of the outer lens 11, and the annular inner wall 23 defining the annular inner groove 20 of the flexible frame 2 is still bonded to the inner annular edge portion 120 on the inner side 121 of the inner lens 12. Thus, water leakage into the interior of the frame 2 is less likely to occur. An improved water resistance is thus provided.

FIG. 4 illustrates a second embodiment of the invention, wherein adhesive 3 may be applied to the periphery 113 of the outer lens 11, the periphery 123 of the inner lens 12, and an annular edge portion 15 between the outer lens 11 and the inner lens 12. The adhesive 3 increases the bonding force for the composite lens 1 and the frame 2 without adversely affecting the vision, as the adhesive 3 is not applied to a central portion between the outer lens 11 and the inner lens 12.

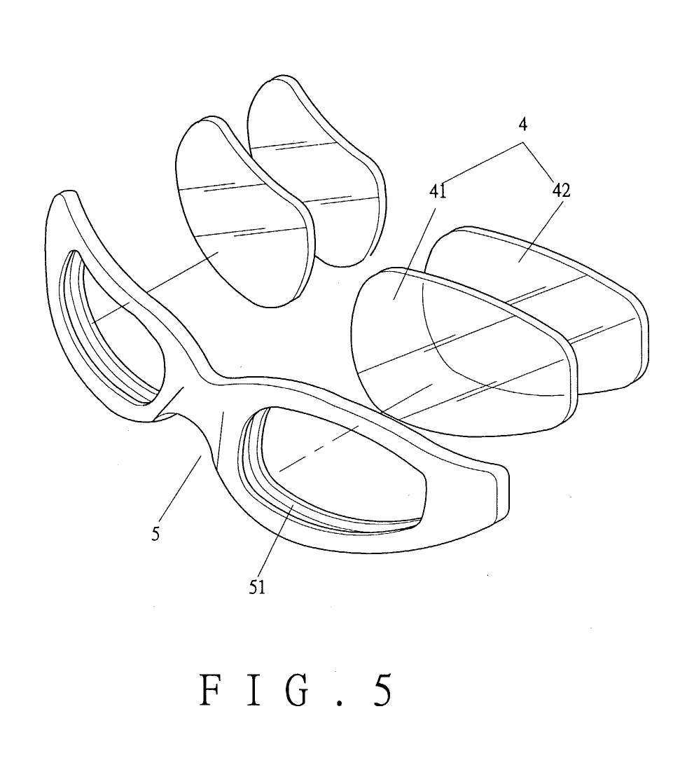

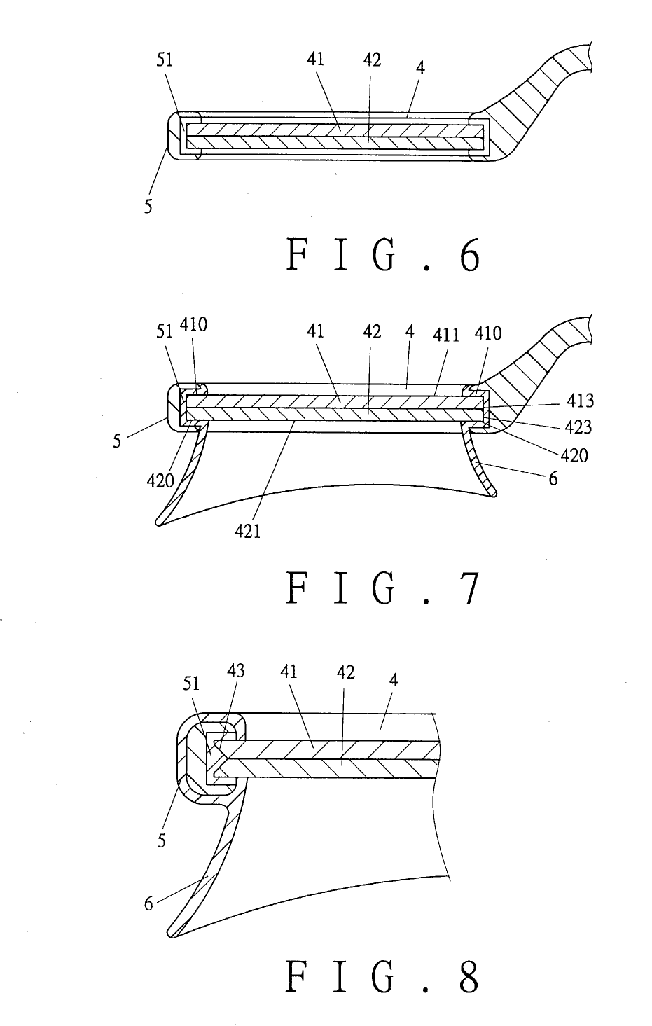

FIGS. 5 through 7 illustrates a third embodiment of the invention. In this embodiment, a composite lens 4 in accordance with the present invention includes a first, outer lens 41 and a second, inner lens 42 that is made from a material different from that for the outer lens 1. The outer lens 41 and the inner lens 42 are placed in a face-to-face manner as that in the first embodiment. A frame 5 includes an annular inner groove 51 for receiving the composite lens 4, with a periphery of the composite lens 4 being received in the annular inner groove 51 of the frame 5.

As illustrated in FIG. 7, the composite lens 4 and the frame 5 are placed into a mold (not shown), and plastic material is poured into the mold to form an integral padding member 6, with the padding member 6 being formed between the composite lens 4 and bonding with a periphery 413 of the outer lens 41 and a periphery 423 of the inner lens 42, an outer annular edge portion 410 on an outer side 411 of the outer lens 41, and an inner annular edge portion 420 on an inner side 421 of the inner lens 42. Thus, the composite lens 4 is integrally formed with the soft padding member 6. The pair of goggles for swimming or diving thus formed provides good vision, as there is no adhesive between the outer lens 41 and the inner lens 42. Even though ambient changes and thus causes deformation of the outer lens 41 and the inner lens 42 made from different materials, the soft padding member 6 is still reliably bonded to the outer lens 41 and the inner lens 42. An improved water resistance is thus improved. Further, the soft padding member 6 provides improved effect to absorb vibrations. Further, since the padding member 6 is integrally formed with the composite lens 4 and the frame 5, leakage of water into an interior of the frame 5 is less likely to occur.

FIG. 8 illustrates a fourth embodiment that is modified from the third embodiment. In this embodiment, the composite lens 4 includes an annular conic groove 43 in a periphery thereof. The material for forming the padding member 6 enters the annular conic groove 43 to increase the bonding strength and to further improve water resistance. The padding member 6 may envelope the frame 5 such that a stable, reliable engagement is provided between the padding member 6, the frame 5, and the composite lens 4.

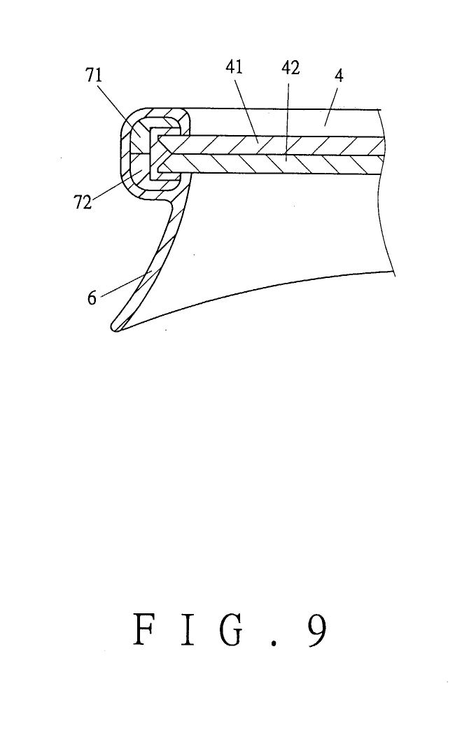

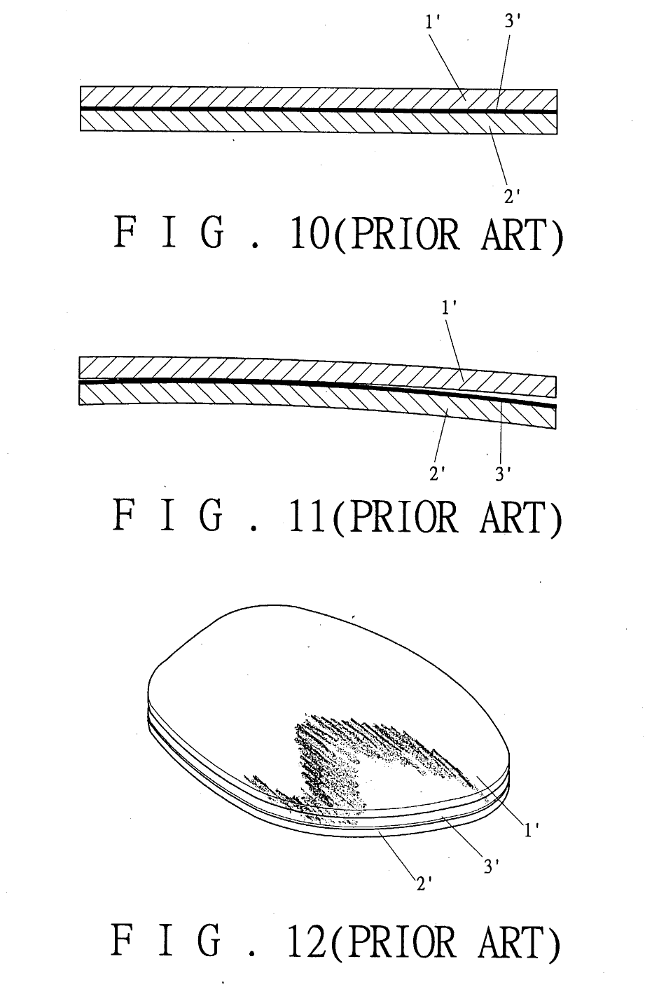

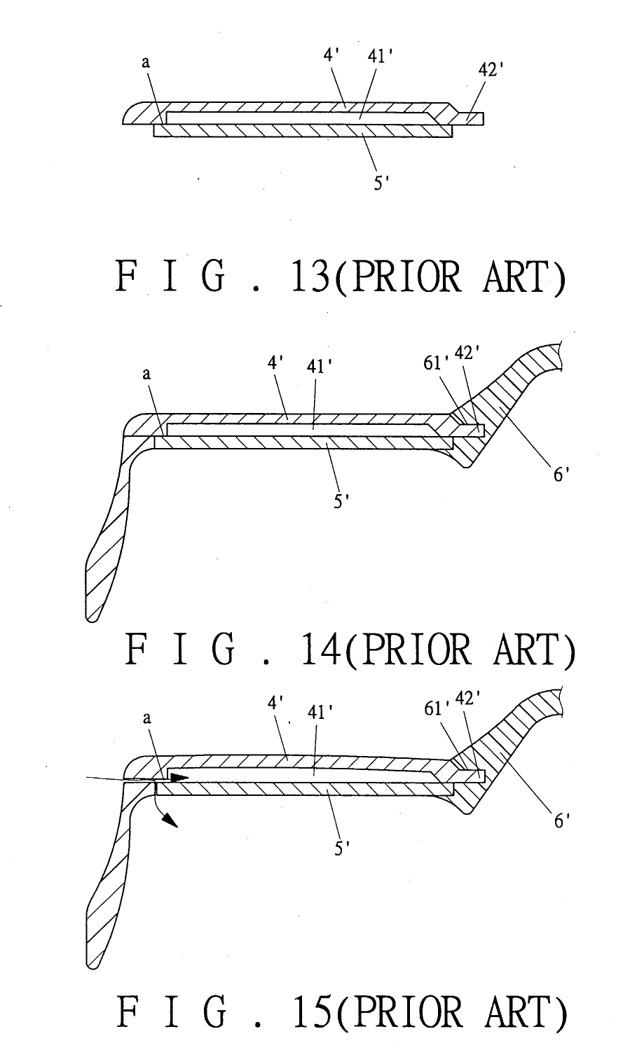

FIG. 9 illustrates a fifth embodiment that is modified from the fourth embodiment of FIG. 8. In this embodiment, the frame consists of two frame halves 71 and 72. The composite lens 4 is mounted into a space between the frame halves 71 and 72, and plastic material is poured into the mold for forming a padding member 6, which is similar to that of the fourth embodiment of FIG. 8.

The composite lens 1, 4 may include more than two lenses without departing from the scope of the invention.

Although the invention has been explained in relation to its preferred embodiments, it is to be understood that many other possible modifications and variations can be made without departing from the scope of the invention as hereinafter claimed.

What is claimed is:

1. A pair of goggles, comprising:
a flexible frame including an annular groove, the annular groove being defined by an annular bottom wall, an annular inner wall, and an annular outer wall; and
a composite lens including a plurality of lenses that are overlapped one on another;
with the annular bottom wall defining the annular inner groove of the flexible frame being bonded with a periphery of each of the plurality of lenses as an integral unit.

2. The pair of goggles as claimed in claim 1, with the annular outer wall defining the annular inner groove of the flexible frame being bonded with an outer annular edge portion on an outer side of an outermost one of the plurality of lenses, and with the annular inner wall defining the annular inner groove of the flexible frame being bonded with an inner annular edge portion on an inner side of an innermost one of the plurality of lenses.

3. The pair of goggles as claimed in claim 2, with adhesive being applied to the periphery of each of the plurality of lenses and an annular edge portion between two of the plurality of lenses adjacent to each other.

4. A pair of goggles, comprising:
a frame including an annular groove;
a composite lens including a plurality of lenses that are overlapped one on another, the composite lens having a periphery received in the frame; and
a soft padding member having a portion mounted between a periphery of the composite lens and the frame;
with the soft padding member being bonded with the frame as an integral unit; and
with the soft padding member being bonded with a periphery of each of the plurality of lenses as an integral unit.

5. The pair of goggles as claimed in claim 4, wherein the composite lens includes an annular conic groove in the periphery thereof.
